(19) United States
(12) Patent Application Publication
(10) Pub. No.: US 2025/0312532 A1
(43) Pub. Date: Oct. 9, 2025

(54) Drug Delivery Device With Fluid Flow Path Sensing

(71) Applicant: Becton, Dickinson and Company, Franklin Lakes, NJ (US)

(72) Inventors: Steve Beguin, Nenzlingen (CH); Sean McGrath, Artane (IE); Simon O'Reilly, Dublin (IE); Stuart Plascott, Foxrock (IE); Christian Sandmann, Wayne, NJ (US)

(21) Appl. No.: 18/864,579

(22) PCT Filed: Jun. 6, 2023
(86) PCT No.: PCT/US23/24576

§ 371 Date: Nov. 11, 2024

Related U.S. Application Data
(60) Provisional application No. 63/349,789, filed on Jun. 7, 2022.

(51) Int. Cl. A61M 5/172; A61M 5/142

(52) U.S. Cl. CPC A61M 5/172; A61M 5/14248; A61M 2205/18; A61M 2205/3331

(57) ABSTRACT

A drug delivery device that includes a sensor and at least one processor in communication with the sensor, where the at least one processor is programmed or configured to receive data associated with a fluid flow path from the sensor, determine a characteristic associated with the fluid flow path based on the data received from the sensor, determine whether the characteristic associated with the fluid flow path satisfies a threshold, and perform an action based on determining that the data associated with the fluid flow path satisfies the threshold. The sensor and processor analyzing data to determine successful deployment of cannula in target tissue and detect removal of cannula from target tissue.

### CROSS-REFERENCE TO RELATED APPLICATION

The present application claims priority to U.S. Provisional Application No. 63/349,789, entitled “Drug Delivery Device with Fluid Flow Path Sensing”, filed Jun. 7, 2022, the entire disclosure of which is hereby incorporated by reference in its entirety.

### BACKGROUND

Wearable injectors (e.g., wearable drug delivery systems, wearable injection devices, wearable automatic injectors, on-body injectors (OBIS), on-body delivery systems (OBDS), etc.) may provide drug therapy to a patient when the patient is at a location that is remote from a clinical facility and/or while being worn discretely under the patient's clothing. A wearable injector can be applied to the patient's skin and may be configured to automatically or based on a user's command, deliver a dose of a drug (e.g., a pharmaceutical composition, a medication, etc.) when the wearable injector is applied to (e.g., positioned on) the patient's skin, such as after a delay of a predetermined time period or immediately upon application of the wearable injector. In some instances, the wearable injector may deliver the drug to the patient for a duration of time. After the wearable injector delivers the pharmaceutical composition to the patient, the patient may subsequently remove and dispose of the device.

In some situations, when a wearable injector is applied to a body of the patient, the wearable injector may move. For example, after the wearable injector is applied to a limb of the patient, the position of the wearable injector may change based on movements of the limb of the patient. In some instances, the wearable injector and components thereof, such as a cannula and/or a needle, may be displaced from an intended position based on movement of the patient. Movement of the wearable injector can disrupt proper introduction of a drug to the patient. In some instances, if extensive movement occurs, components of the wearable injector may not be deployed properly, interrupting delivery of the drug to the patient. Furthermore, movement of the wearable injector may disrupt a fluid flow path from a reservoir of the wearable injector to the injection site of the patient.

Accordingly, it is beneficial to determine whether components of a wearable injector are properly deployed with regard to a body of a patient and whether a fluid flow path is functioning properly (e.g., whether a fluid flow path is not obstructed, whether a fluid flow path is not leaking, whether an indwelling catheter is deployed in subcutaneous tissue, etc.) to ensure correct delivery of a drug to the patient.

### SUMMARY

Accordingly, provided are improved systems, devices, products, apparatus, and/or methods for sensing a characteristic of a fluid flow path of a drug delivery device.

Further non-limiting embodiments or aspects are set forth in the following numbered clauses:

Clause 1. A drug delivery device, for delivery of a drug, including at least one sensor, and at least one processor in communication with the sensor. The at least one processor is programmed or configured to receive data associated with a fluid flow path from the sensor during a time corresponding to a working mode of the drug delivery device, determine a characteristic associated with the fluid flow path based on the data received from the sensor, determine whether the characteristic associated with the fluid flow path satisfies at least one of a threshold and a characteristic time constant derived from a pressure decay, and perform an action based on determining that the data associated with the fluid flow path satisfies the threshold at a time prior to, or upon completion of, delivery of a drug from the drug delivery device.

Clause 2. A drug delivery device of clause 1, wherein the sensor is a flow sensor.

Clause 3. A drug delivery device of clause 1, wherein the sensor is a pressure sensor.

Clause 4. A drug delivery device of clause 3, wherein the pressure sensor is positioned within the fluid flow path.

Clause 5. A drug delivery device of clause 1, wherein the at least one sensor is positioned in direct contact with the fluid path.

Clause 6. A drug delivery device of clause 1, further including a housing, and a drug injection system. The at least one processor and the drug injection system are positioned within the housing.

Clause 7. A drug delivery device of clause 6, wherein the drug injection system includes a cannula configured to be inserted into subcutaneous tissue or muscle tissue of a user.

Clause 8. A drug delivery device of clause 1, wherein the threshold includes a threshold value based on a working mode of the drug delivery device.

Clause 9. A drug delivery device of clause 1, wherein the specific working mode of the drug delivery device is at least one of deployment of a component, injection of a drug, and dwell time.

Clause 10. A drug delivery device including a housing, a drug injection system, a sensor, and at least one processor in communication with the sensor. The at least one processor, the sensor, and the drug injection system are positioned within the housing, and wherein the at least one processor is programmed or configured to receive data associated with a fluid flow path from the sensor, determine a characteristic associated with the fluid flow path based on the data received from the sensor, determine whether the characteristic associated with the fluid flow path satisfies a threshold, and perform an action based on determining that the data associated with the fluid flow path satisfies the threshold at a time prior to, or upon completion of, delivery of a drug from the drug delivery device.

Clause 11. A drug delivery device of clause 10, wherein the sensor is a flow sensor.

Clause 12. A drug delivery device of clause 10, wherein the sensor is a pressure sensor.

Clause 13. A drug delivery device of clause 12, wherein the pressure sensor is positioned within the fluid flow path.

Clause 14. A drug delivery device of clause 10, wherein the at least one sensor is positioned within the fluid path.

Clause 15. A drug delivery device of clause 10, further including a housing, and a drug injection system. The at least one processor and the drug injection system are positioned within the housing.

Clause 16. A drug delivery device of clause 15, wherein the drug injection system includes a cannula configured to be inserted into subcutaneous tissue or muscle tissue of a user.

Clause 17. A drug delivery device of clause 10, wherein the threshold includes a threshold value based on a working mode of the drug delivery device.

Clause 18. A drug delivery device includes at least one processor programmed or configured to receive data associated with a fluid flow path from a sensor, determine a characteristic associated with the fluid flow path based on the data received from the sensor, determine whether the characteristic associated with the fluid flow path satisfies a threshold, and perform an action based on determining that the data associated with the fluid flow path satisfies the threshold. When performing the action, the at least one processor is programmed or configured to provide an alarm, provide a notification, store data associated with an event, or any combination thereof.

Clause 19. A drug delivery device of clause 18, wherein the sensor is positioned within the fluid flow path.

Clause 20. A drug delivery device of clause 18, further including a housing, a reservoir, a pump device, a drug injection system, and an adhesive layer. The at least one processor, the reservoir, the pump device, and the drug injection system are positioned within the housing, and the adhesive layer is configured to be positioned on a body of a user.

Clause 21. A drug delivery device of clause 20, wherein the drug injection system comprises a cannula configured to be inserted into subcutaneous tissue or muscle tissue of a user.

Clause 22. A drug delivery device of clause 18, wherein the action is performed based on determining that the data associated with the fluid flow path satisfies the threshold at a time prior to, or upon completion of, delivery of a drug from the drug delivery device.

### BRIEF DESCRIPTION OF THE DRAWINGS

Additional advantages and details are explained in greater detail below with reference to the exemplary embodiments that are illustrated in the accompanying schematic figures, in which:

FIG. 1 is a diagram of a non-limiting embodiment of a drug delivery device;

FIG. 2 is a diagram of a non-limiting embodiment of components of one or more devices and/or one or more systems of FIG. 1;

FIG. 3 is a flowchart of a non-limiting embodiment of a process for sensing a characteristic of a fluid flow path of a drug delivery device;

FIG. 4 is a diagram of a non-limiting embodiment of a drug delivery device;

FIG. 5 is a diagram of a non-limiting embodiment of a drug delivery device;

FIG. 6 is a perspective view of a non-limiting embodiment of a drug delivery device;

FIG. 7 is a cross-sectional view of the drug delivery device shown in FIG. 6;

FIG. 8 is a perspective view of the drug delivery device of FIG. 6, with a housing removed;

FIG. 9 is a schematic of the drug delivery device of FIG. 6;

FIG. 10 is a diagram of a non-limiting embodiment of an implementation of a drug injection system of a drug delivery device during an injection procedure;

FIG. 11 is a diagram of a non-limiting embodiment of a base section of a drug delivery device;

FIG. 12 is an illustration of a non-limiting embodiment of a sensor device;

FIG. 13 is a diagram of a non-limiting embodiment of the sensor device shown in FIG. 12 positioned within the base section shown in FIG. 11; and

FIG. 14 is a bottom view of a non-limiting embodiment of a drug delivery device.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” As used in the specification and the claims, the singular form of “a,” “an,” and “the” include plural referents, such as unless the context clearly dictates otherwise. Additionally, Furthermore, as used herein, the terms “set” and “group” are intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or send (e.g., transmit) information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and transmits the processed information to the second unit. In some non-limiting embodiments or aspects, a message may refer to a network packet (e.g., a data packet and/or the like) that includes data.

Some non-limiting embodiments or aspects may be described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Provided are improved devices, systems, methods and computer program products for sensing a characteristic of a fluid flow path of a drug delivery device. Embodiments of the present disclosure may include a drug delivery device that includes a sensor and at least one processor in communication with the sensor, wherein the at least one processor is programmed or configured to receive data associated with a fluid flow path from the sensor, determine a characteristic associated with the fluid flow path based on the data received from the sensor, determine whether the characteristic associated with the fluid flow path satisfies a threshold, and perform an action based on determining that the data associated with the fluid flow path satisfies the threshold. In some non-limiting embodiments, when performing the action, the at least one processor is programmed or configured to provide an alarm, provide a notification, store data associated with an event, and/or cause an operation to stop.

In some non-limiting embodiments, the threshold comprises a threshold value based on a working mode of the drug delivery device. In some non-limiting embodiments, the at least one processor is further programmed or configured to determine the proper deployment of an indwelling catheter in the subcutaneous tissue by mean of measurement of a vacuum pressure below a threshold and with a characteristic time constant for the pressure decay comprised within a defined range, when determining the characteristic associated with the fluid flow path, the at least one processor is programmed or configured to determine the characteristic associated with the fluid flow path based on the vacuum measured of the sensor. In some non-limiting embodiments, a pressure pattern or fluid movement pattern characteristic of catheter withdrawal from tissue is measured by the sensor and analyzed by the processor to trigger an action such as an alarm or notification or data logging event. In some non-limiting embodiments, the at least one processor and the sensor are positioned on a printed circuit board (PCB). In some non-limiting embodiments, the drug delivery device further comprises a housing and a drug injection system, and the at least one processor and the drug injection system are positioned within the housing. In some non-limiting embodiments, the drug injection system comprises a cannula configured to be inserted into subcutaneous tissue, dermis, or muscle tissue of a user (e.g., a patient).

In this way, embodiments of the present disclosure allow for a wearable drug delivery device that may determine whether components of a wearable injector are properly deployed with regard to a body of a patient and whether a fluid flow path is functioning properly to ensure correct delivery of a drug to the patient.

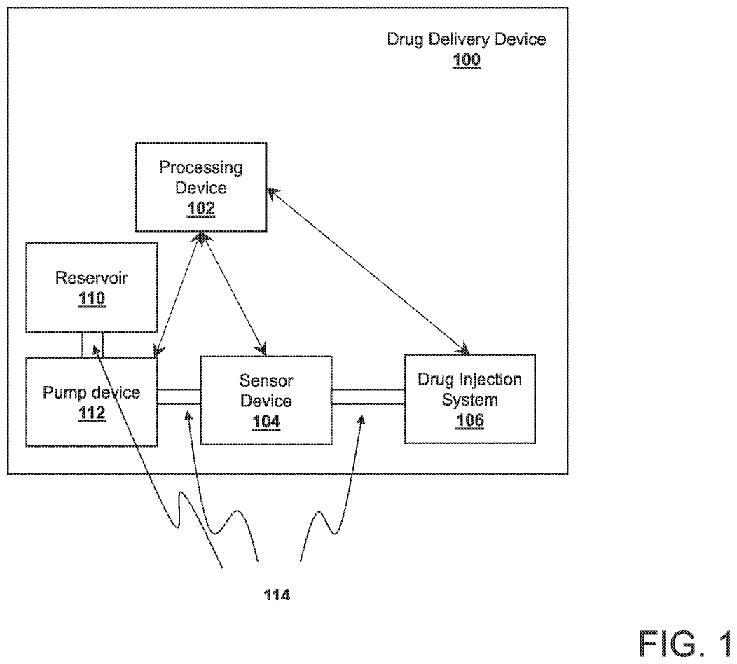

Referring now to FIG. 1, FIG. 1 is a diagram of drug delivery device 100. As shown in FIG. 1, drug delivery device 100 may include processing device 102, sensor device 104, drug injection system 106, reservoir 110, and pump device 112. Processing device 102, sensor device 104, drug injection system 106, reservoir 110, and pump device 112 may interconnect (e.g., establish a connection to communicate, and/or the like) via wired connections, wireless connections, or a combination of wired and wireless connections.

In some non-limiting embodiments, drug delivery device 100 may be a wearable automatic injector, such as an insulin or bone marrow stimulant delivery device. For example, drug delivery device 100 may be applied to the skin of a user and triggered to inject a drug into the user. Drug delivery device 100 may be pre-filled with the drug, or it may be filled (e.g., refilled following a prior injection) with the drug by the user or another individual (e.g., a medical professional) prior to use.

Drug delivery device 100 may be configured to deliver a dose of a drug into the body of a user by a subcutaneous injection at a controlled rate of delivery. The controlled rate of delivery may include time durations for the delivery from about 5 minutes to about 60 minutes, but are not limited to this exemplary range. Exemplary volumes of the drug delivered by drug delivery device 100 may range from about 0.1 milliliters to about 10 milliliters, but are not limited to this range. The volume of the drug delivered to the user may be adjusted (e.g., atomically adjusted or manually adjusted).

In some non-limiting embodiments, processing device 102 may include a device that is capable of being in communication with sensor device 104 and/or drug injection system 106. In some non-limiting embodiments, processing device 102 may include a device controller (e.g., a microcontroller), a processor, an integrated circuit, and/or the like. In some non-limiting embodiments, processing device 102 may provide commands (e.g., instructions, signals, etc.) to drug injection system 106 to cause drug injection system 106 to deliver a drug to a body of a user (e.g., a patient) upon which drug delivery device 100 is applied. In some non-limiting embodiments, processing device 102 may provide commands (e.g., instructions, signals, etc.) to pump device 112 to cause pump device 112 to provide a drug from reservoir 110 to fluid flow path 114, so that the drug may be delivered to the body of the user upon which drug delivery device 100 is applied.

In some non-limiting embodiments, sensor device 104 may include a device that is capable of being in communication with processing device 102. In some non-limiting embodiments, sensor device 104 may include a sensor, such as a fluid flow sensor, a pressure sensor, a temperature, and/or a combined pressure and temperature sensor. In some non-limiting embodiments, sensor device 104 may provide data associated with a fluid flow path (e.g., data associated with a flow rate of fluid in the fluid flow path, data associated with a pressure of the fluid flow path (e.g., a pressure within the fluid flow path), data associated with a temperature measurement in the fluid flow path, data associated with a temperature measurement of a fluid in the fluid flow path, etc.) based on sensor device 104 sensing (e.g., measuring) a characteristic of the fluid flow path.

In some non-limiting embodiments, drug injection system 106 may include a device that is capable of being in communication with processing device 102. In some non-limiting embodiments, drug injection system 106 may include a device (e.g., a mechanism, a structure, etc.) that is capable of delivering a drug to a body of a user upon which drug delivery device 100 is applied. In some non-limiting embodiments, drug injection system 106 may automatically deliver the drug to the body of the user according to one or more predetermined time intervals. Additionally or alternatively, drug injection system 106 may automatically deliver the drug to the body of the user at a predetermined time period, such as at a predetermined time period following application of drug delivery device 100 to the body of the user. In some non-limiting embodiments, drug injection system 106 may include a cannula, a catheter, and/or a needle that is configured to be positioned within the body of the user. For example, drug injection system 106 may include a cannula, a catheter, and/or a needle that is configured to be inserted into subcutaneous tissue or muscle tissue of the user.

In some non-limiting embodiments, reservoir 110 may include a container (e.g., a bladder, a capsule, etc.) that stores a drug that is intended to be delivered to a body of a user. In some non-limiting embodiments, the container may be refillable. In some non-limiting embodiments, the container may be configured to be removable and/or replaceable. In some non-limiting embodiments, the container may not be refillable (e.g., the container may be a single-use container). In some non-limiting embodiments, the container may not be configured to be removable and/or replaceable.

In some non-limiting embodiments, pump device 112 may include a device that is capable of being in communication with processing device 102. In some non-limiting embodiments, pump device 112 may include a pump (e.g., a positive-displacement pump, an impulse pump, a velocity pump, a gravity pump, a valveless pump, etc.) that moves a fluid (e.g., a drug stored in reservoir 110) based on a mechanical action. In some non-limiting embodiments, pump device 112 may include a power source that allows for operation of pump device 112.

In some non-limiting embodiments, fluid flow path 114 may include any appropriate structure for allowing a fluid to flow along a defined path in drug delivery device 100 and to a body of a user. In some non-limiting embodiments, fluid flow path 114 may include a defined channel. In some non-limiting embodiments, fluid flow path 114 may include a tube (e.g., tubing, a hose, etc.). In some non-limiting embodiments, fluid flow path 114 may include a catheter, a cannula, and/or a needle (e.g., of drug injection system 106) that extends outside of drug delivery device 100 (e.g., extends outside of drug delivery device 100 and into the body of the user).

In some non-limiting embodiments, processing device 102, sensor device 104, drug injection system 106, reservoir 110, and/or pump device 112 may be positioned on (e.g., installed on, placed on, integrated with, etc.) a base section (e.g., a baseplate, a bottom plate, etc.) or a printed circuit board (PCB) of drug delivery device 100. For example, one or more of processing device 102, sensor device 104, drug injection system 106, reservoir 110, and/or pump device 112 may be positioned on the same base section or PCB. In some non-limiting embodiments, the PCB that has processing device 102, sensor device 104, drug injection system 106, reservoir 110, and/or pump device 112 positioned thereon, may be positioned within a housing of drug delivery device 100. Additionally or alternatively, the base section that has processing device 102, sensor device 104, drug injection system 106, reservoir 110, and/or pump device 112 positioned thereon, may be positioned with the housing of drug delivery device 100 covering processing device 102, sensor device 104, drug injection system 106, reservoir 110, and/or pump device 112 that is positioned thereon. In this way, drug delivery device 100 may have a compact form factor that allows for a user to wear drug delivery device 100 with minimal discomfort.

In some non-limiting embodiments, drug delivery device 100 may include a mechanism for holding drug delivery device 100 in contact with a body of a user. In this way, drug delivery device 100 may be a body worn device. In one example, drug delivery device 100 may include an adhesive layer that has an adhesive appropriate for holding drug delivery device 100 on the body of the user for an extend time period. Additionally or alternatively, drug delivery device 100 may include a band (e.g., a strap, a belt, a wrap, etc.) configured to hold drug delivery device 100 in contact with the body of the user. In some non-limiting embodiments, the adhesive layer may be positioned on a surface of drug delivery device 100 that is positioned near the body of the user when drug delivery device 100 is applied to the body of the user. Further, a component of drug injection system 106 may be configured to extend through the adhesive layer to be positioned within the body of the user. In some non-limiting embodiments, the component of drug injection system 106 may be configured to extend through an aperture of the adhesive layer. In some non-limiting embodiments, the component of drug injection system 106 may be configured to penetrate through the adhesive layer.

In some non-limiting embodiments, drug delivery device 100 may include a device for providing an indication associated with an operation (e.g., an indication of proper operation, an indication of improper operation, an indication that an operation has been stopped, etc.) of drug delivery device 100. For example, drug delivery device 100 may include one or more devices that provide a visual prompt to a user, such as one or more light emitting diodes (LEDS) and/or one or more display screens. Additionally or alternatively, drug delivery device 100 may include one or more devices that provide an audible prompt to a user, such as one or more electroacoustic transducers (e.g., one or more speakers).

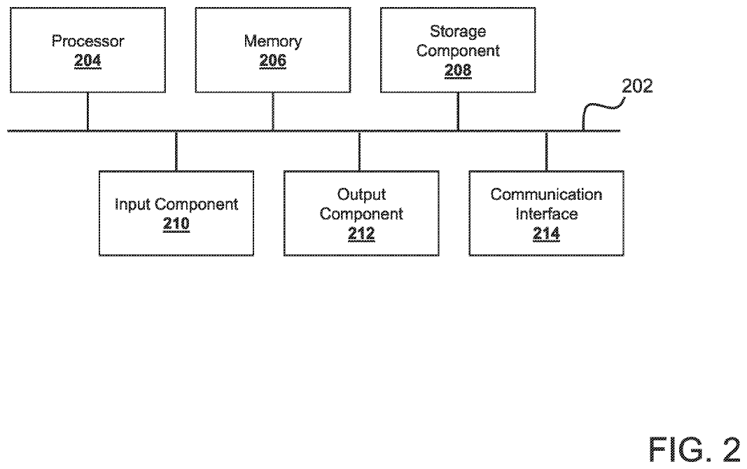

Referring now to FIG. 2, FIG. 2 is a diagram of example components of device 200. Device 200 may correspond to processing device 102, sensor device 104, and/or one or more components of drug injection system 106. In some non-limiting embodiments, processing device 102, sensor device 104, and/or drug injection system 106 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a Bluetooth® interface, a Zigbee® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database and/or the like). Device 200 may be capable of receiving information from, storing information in, communicating information to, or searching information stored in the data storage or one or more data structures in memory 206 and/or storage component 208. For example, the information may include input data, input data, output data, transaction data, account data, or any combination thereof.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

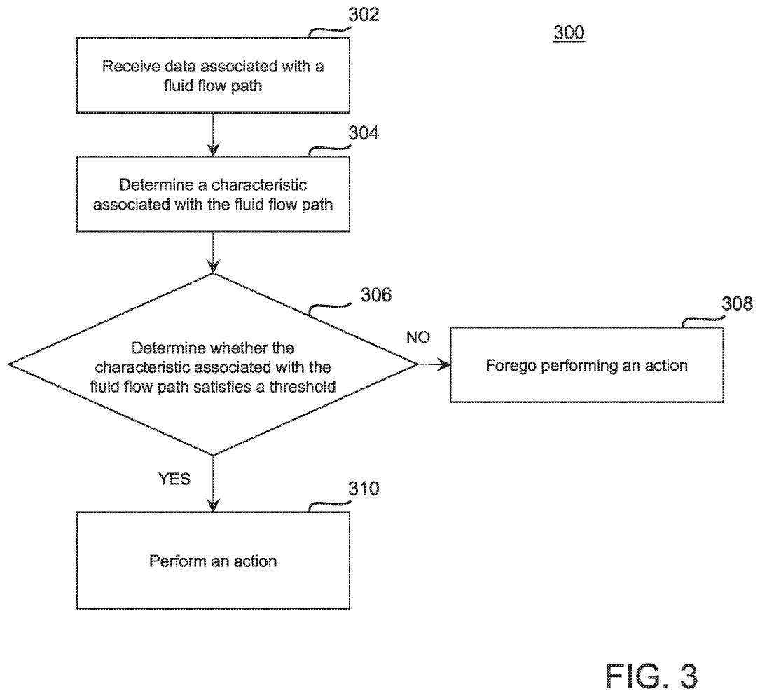

Referring now to FIG. 3, FIG. 3 is a flowchart of a non-limiting embodiment or aspect of process 300 for sensing a characteristic of a fluid flow path of a drug delivery device. In some non-limiting embodiments or aspects, one or more of the functions described with respect to process 300 may be performed (e.g., completely, partially, etc.) by processing device 102. In some non-limiting embodiments or aspects, one or more of the steps of process 300 may be performed (e.g., completely, partially, and/or the like) by another device or a group of devices separate from and/or including processing device 102, such as sensor device 104, drug injection system 106, reservoir 110, and/or pump device 112.

As shown in FIG. 3, at step 302, process 300 may include receiving data associated with a fluid flow path. For example, processing device 102 may receive the data associated with fluid flow path 114 from sensor device 104. In some non-limiting embodiments, the data associated with fluid flow path 114 may include any information (e.g., a characteristic, a parameter, etc.) regarding fluid flow path 114 that is determined (e.g., measured, detected, sensed, obtained, etc.) by sensor device 104 in the course of operation of sensor device 104. In some non-limiting embodiments, the information regarding fluid flow path 114 may include data associated with a flow rate of fluid in fluid flow path 114, data associated with a pressure of fluid flow path 114, such as a pressure within fluid flow path 114, data associated with a temperature measurement in fluid flow path 114, data associated with a temperature measurement of a fluid in fluid flow path 114, and/or the like.

As shown in FIG. 3, at step 304, process 300 may include determining a characteristic associated with the fluid flow path. For example, processing device 102 may determine the characteristic associated with fluid flow path 114 based on the data received from sensor device 104. In some non-limiting embodiments, processing device 102 may determine an orientation of sensor device 104. In some non-limiting embodiments, processing device 102 may calibrate sensor device 104 based on the orientation of sensor device 104. In some non-limiting embodiments, processing device 102 may determine the characteristic associated with fluid flow path 114 based on the orientation of sensor device 104 and/or a calibration of sensor device 104.

As shown in FIG. 3, at step 306, process 300 may include determining whether the characteristic associated with the fluid flow path satisfies a threshold. For example, processing device 102 may determine whether the characteristic associated with fluid flow path 114 satisfies a threshold. In some non-limiting embodiments, the threshold may include a threshold value that is based on a flow rate of a fluid and/or a threshold value of pressure. In one example, the threshold may include a threshold value that is based on a flow rate that indicates that a fluid is flowing at an acceptable flow rate (e.g., a maximum acceptable flow rate, a minimum acceptable flow rate, etc.) within fluid flow path 114. Additionally or alternatively, the threshold may include a threshold value that is based on a pressure that indicates that a pressure of fluid flow path 114 is an acceptable pressure (e.g., a maximum acceptable pressure, a minimum acceptable pressure, etc.). In some non-limiting embodiments, the threshold may include a plurality of threshold values (e.g., a profile of threshold values) that include a plurality of predetermined values of flow rate and/or a plurality of predetermined values of pressure. For example, the threshold may include a plurality of predetermined values of flow rate and/or a plurality of predetermined values of pressure at time intervals following deployment of a cannula and/or needle of drug injection system 106. In some non-limiting embodiments, the threshold may include a plurality of predetermined values of flow rate and/or a plurality of predetermined values of pressure based on an amount of a drug stored in reservoir 110 and/or an operation of pump device 112 to provide the amount of the drug stored in reservoir 110 to fluid flow path 114.

In some non-limiting embodiments, the threshold may include a threshold value that is based on a working mode of drug delivery device 100. Drug delivery device 100 may have a plurality of working modes, including a mode associated with a period when a user is asleep (e.g., deep sleep), a working mode associated with preparation of applying drug delivery device 100 to a body of a user, a working mode associated with deployment of a component (e.g., a cannula, a catheter, and/or a needle) of drug injection system 106, and/or a working mode associated with an injection of a drug (e.g., a time at which the drug is being delivered) to the body of the user, a working mode associated with the period of dwell of the device with the patient. In some non-limiting embodiments, the threshold value may be based on the working mode, such that a threshold value for a first working mode may be different than a threshold value for a second working mode. In some non-limiting embodiments, the threshold value may be different for each working mode of the plurality of working modes of drug delivery device 100.

As shown in FIG. 3, at step 310 (“YES”), process 300 may include performing an action based on determining that the characteristic associated with the fluid flow path satisfies the threshold. For example, processing device 102 may perform an action based on determining that the characteristic associated with fluid flow path 114 satisfies the threshold. In some non-limiting embodiments, processing device 102 may compare the characteristic associated with fluid flow path 114 to the threshold (e.g., a threshold value), and, if processing device 102 determines that the characteristic associated with fluid flow path 114 satisfies the threshold, processing device 102 may perform the action. In some non-limiting embodiments, processing device 102 may perform the action by providing an alarm. For example, processing device 102 may cause (e.g., trigger) an alarm to be emitted by drug delivery device 100. In some non-limiting embodiments, processing device 102 may perform the action by providing a notification. For example, processing device 102 may cause a notification (e.g., a notification based on a visual prompt, such as a light emitting diode being illuminated, a notification based on an audible prompt, such as a speaker emitting an audible sound, etc.) to be provided by drug delivery device 100. In some non-limiting embodiments, processing device 102 may perform the action by storing (e.g., logging) data associated with an event (e.g., an event indicating that the characteristic associated with fluid flow path 114 satisfies a threshold). For example, processing device 102 may store data associated with the event in a memory device of drug delivery device 100. In some non-limiting embodiments, processing device 102 may perform the action by causing pump device 112 and/or drug injection system 106 to stop an operation.

As shown in FIG. 3, at step 308 (“NO”), process 300 may include foregoing performing an action based on determining that the characteristic associated with the fluid flow path does not satisfy the threshold. For example, processing device 102 may forego performing an action based on determining that the characteristic associated with fluid flow path 114 does not satisfy the threshold. In some non-limiting embodiments, processing device 102 may compare the characteristic associated with fluid flow path 114 to the threshold, and, if processing device 102 determines that the characteristic associated with fluid flow path 114 does not satisfy the threshold, processing device 102 may forego performing the action.

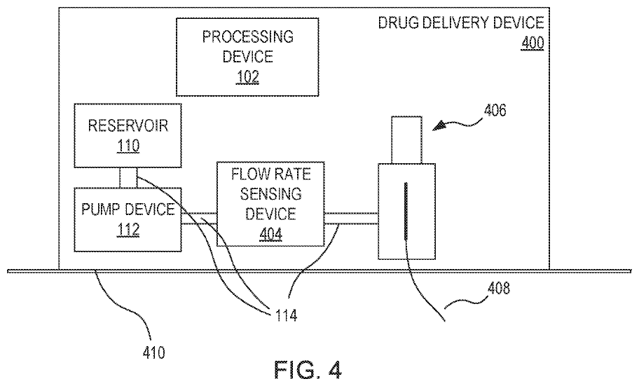

Referring now to FIG. 4, FIG. 4 is a diagram of drug delivery device 400. In some non-limiting embodiments, drug delivery device 400 may be the same as or similar to drug delivery device 100. As shown in FIG. 4, drug delivery device 400 may include processing device 102, flow rate sensing device 404, reservoir 110, pump device 112, drug injection system 406, and adhesive layer 410. In some non-limiting embodiments, flow rate sensing device 404 may be the same as or similar to sensor device 104. In some non-limiting embodiments, drug injection system 406 may include cannula 408. In some non-limiting embodiments, cannula 408 may include a needle. In some non-limiting embodiments, cannula 408 may include a catheter, which may be a soft, flexible catheter, a rigid catheter, or a combination thereof. In some non-limiting embodiments, adhesive layer 410 may include a structural layer (e.g., a base section, such as a baseplate, etc.) and an adhesive applied on the structural layer. In some non-limiting embodiments, the structural layer may include a flexible polymeric foam, a woven material, a hard plastic material, and/or the like. In some non-limiting embodiments, adhesive layer 410 may come into contact with the body of a user. For example, adhesive layer 410 may be applied to the skin of a user. In some non-limiting embodiments, cannula 408 and/or a needle of cannula 408 may be inserted into the skin of a user.

With regard to drug delivery device 400, processing device 102 may be programmed or configured to perform a process that is the same or similar to process 300 for sensing a characteristic of a fluid flow path of drug delivery device 400 based on flow rate sensing device 404. In some non-limiting embodiments, processing device 102 may receive data associated with fluid flow path 114 from flow rate sensing device 404. In some non-limiting embodiments, the data associated with fluid flow path 114 may include data associated with a fluid flowing in fluid flow path 114. For example, the data associated with fluid flow path 114 may include a flow rate of a fluid flowing in fluid flow path 114.

In some non-limiting embodiments, processing device 102 may determine a characteristic associated with fluid flow path 114 based on the data associated with fluid flow path 114. For example, processing device 102 may determine a flow rate of a fluid flowing in fluid flow path 114 based on the data associated with fluid flow path 114.

In some non-limiting embodiments, processing device 102 may determine whether the characteristic associated with fluid flow path 114 satisfies a threshold value based on the characteristic associated with fluid flow path 114. For example, processing device 102 may determine whether the flow rate of a fluid flowing in fluid flow path 114 satisfies a threshold value of flow rate.

In some non-limiting embodiments, processing device 102 may perform an action based on determining that the characteristic associated with fluid flow path 114 satisfies the threshold value or forego performing an action based on determining that the characteristic associated with fluid flow path 114 does not satisfy the threshold value. In some non-limiting embodiments, processing device 102 may perform the action based on determining that the flow rate of a fluid flowing in fluid flow path 114 satisfies the threshold value of the flow rate or processing device 102 may forego performing the action based on determining that the flow rate of a fluid flowing in fluid flow path 114 does not satisfy the threshold value of the flow rate.

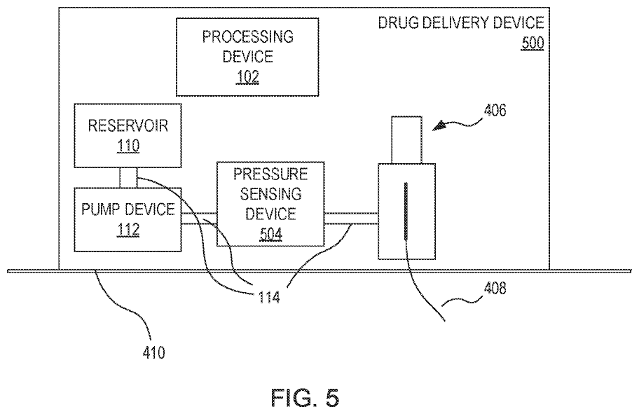

Referring now to FIG. 5, FIG. 5 is a diagram of drug delivery device 500. In some non-limiting embodiments, drug delivery device 500 may be the same as or similar to drug delivery device 100 and/or drug delivery device 400. As shown in FIG. 5, drug delivery device 500 may include processing device 102, pressure sensing device 504, drug injection system 406, and adhesive layer 410. In some non-limiting embodiments, pressure sensing device 504 may be the same as or similar to sensor device 104. In some non-limiting embodiments, pressure sensing device 504 may include a device configured to detect a pressure of (e.g., a pressure within) fluid flow path 114.

With regard to drug delivery device 500, processing device 102 may be programmed or configured to perform a process that is the same or similar to process 300 for sensing a characteristic of a fluid flow path of drug delivery device 500 based on pressure sensing device 504. In some non-limiting embodiments, processing device 102 may receive data associated with fluid flow path 114 from pressure sensing device 504. In some non-limiting embodiments, the data associated with fluid flow path 114 may include data associated with a pressure of fluid flow path 114. For example, the data associated with fluid flow path 114 may include a pressure measured within fluid flow path 114 (e.g., a pressure measured within fluid flow path 114 when a fluid is flowing in fluid flow path 114).

In some non-limiting embodiments, processing device 102 may determine a characteristic associated with fluid flow path 114 based on the data associated with fluid flow path 114. For example, processing device 102 may determine a pressure of fluid flow path 114 based on the data associated with fluid flow path 114.

In some non-limiting embodiments, processing device 102 may determine whether the characteristic associated with fluid flow path 114 satisfies a threshold value based on the characteristic associated with fluid flow path 114. For example, processing device 102 may determine whether the pressure of fluid flow path 114 satisfies a threshold value of pressure.

In some non-limiting embodiments, processing device 102 may perform an action based on determining that the characteristic associated with fluid flow path 114 satisfies the threshold value or forego performing an action based on determining that the characteristic associated with fluid flow path 114 does not satisfy the threshold value. The threshold value may be a static value, a dynamic change of pressure value, a characteristic time constant (e.g. a pressure decay time constant), or a combination thereof. In some non-limiting embodiments, processing device 102 may perform the action based on determining that the pressure of fluid flow path 114 satisfies the threshold value of pressure or processing device 102 may forego performing the action based on determining that pressure of fluid flow path 114 does not satisfy the threshold value of pressure.

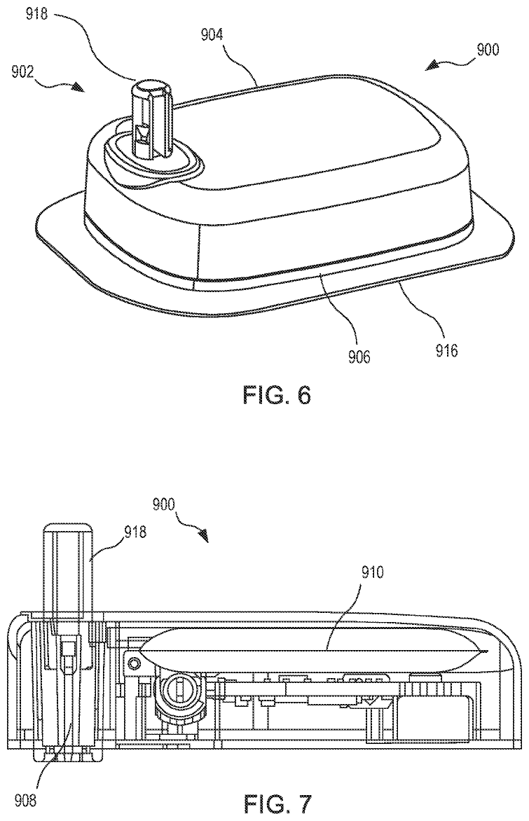

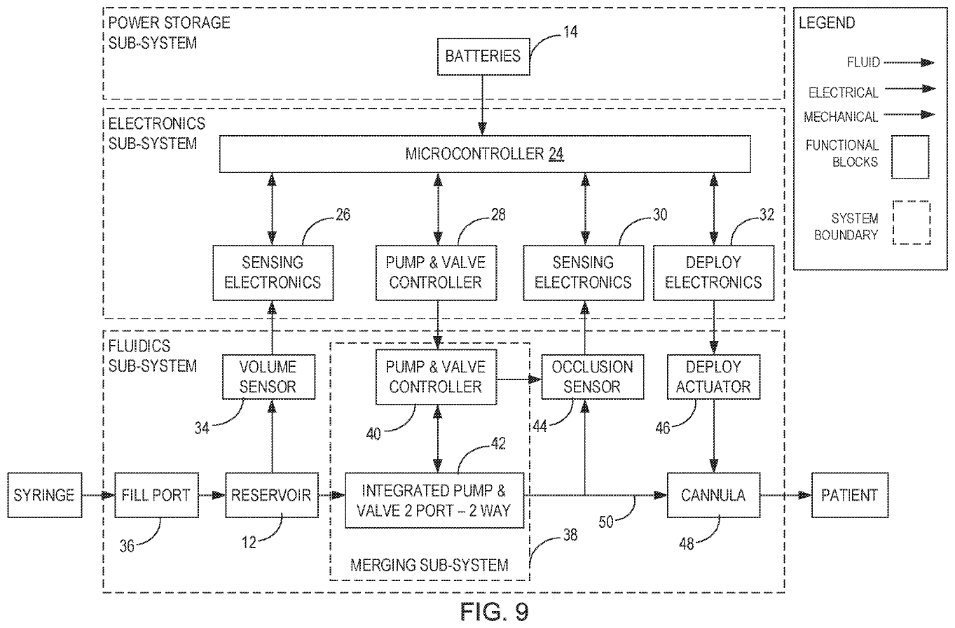

Referring now to FIGS. 6-8, FIGS. 6-8 are illustrations of drug delivery device 900. In some non-limiting embodiments, drug delivery device 900 may be the same or similar to drug delivery device 100, drug delivery device 400, and/or drug delivery device 500. With regard to FIG. 6, FIG. 6 is a perspective view of drug delivery device 900. As shown in FIG. 9, drug delivery device 900 may include drug injection system 902, housing 904, base 906, and button 918. In some non-limiting embodiments, drug delivery device 900 may be a wearable automatic injector. For example, drug delivery device 900 may be an insulin or bone marrow stimulant delivery device. In some non-limiting embodiments, drug delivery device 900 may be applied to (e.g., positioned in contact with) the body of a user. For example, drug delivery device 900 may be mounted onto the skin of a user. In some non-limiting embodiments, base 906 may include adhesive layer 916. In some non-limiting embodiments, adhesive layer 916 may be the same as or similar to adhesive layer 410. In some non-limiting embodiments, button 918 may be configured to allow for manual operation of drug injection system 902, such that a drug may be delivered to the body of the user based on the user depressing button 918.

With regard to FIG. 7, FIG. 7 is a cross-sectional view of drug delivery device 900. As shown in FIG. 7, drug delivery device 900 may include cannula 908 and reservoir 910. In some non-limiting embodiments, cannula 908 may include a needle. In some non-limiting embodiments, cannula 908 may include a catheter, which may be a soft, flexible catheter, a rigid catheter, or a combination thereof. In some non-limiting embodiments, cannula 908 may be inserted into the skin of a user. For example, cannula 908 may be inserted into the skin of a user and a needle of cannula 908 may extend into a body of the user. In some non-limiting embodiments, reservoir 910 may include a drug that is to be delivered (e.g., injected) into the body of the user.

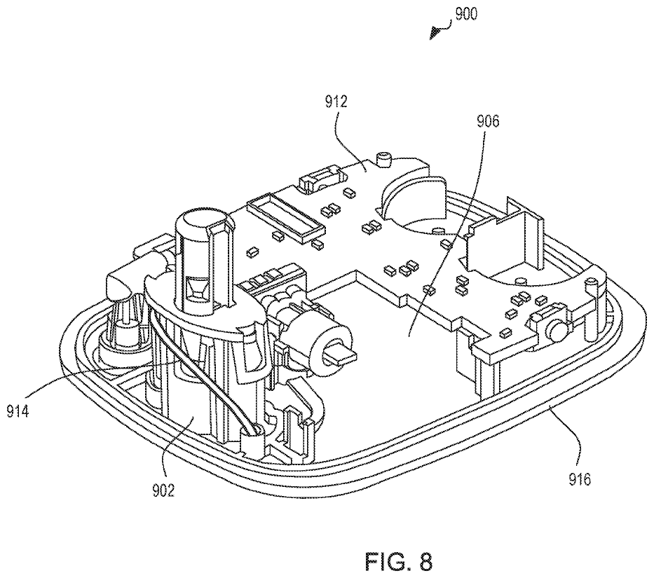

With regard to FIG. 8, FIG. 8 is a perspective view of drug delivery device 900 with housing 904 removed. As shown in FIG. 8, drug delivery device 900 may include drug injection system 902, base 906, printed circuit board 912, and fluid line 914. In some non-limiting embodiments, printed circuit board 912 may include control electronics (e.g., processing device 102) for controlling operation of drug delivery device 900. In some non-limiting embodiments, printed circuit board 912 may include a sensor (e.g., sensor device 104, flow rate sensing device 404, pressure sensing device 504, etc.), drug injection system 902, and/or a processing device (e.g., processing device 102) attached thereon.

Referring now to FIG. 9, FIG. 9 is a schematic diagram of drug delivery device 900 shown in FIG. 6. As shown in FIG. 9, drug delivery device 900 may include a power storage sub-system, an electronics sub-system, and a fluidics sub-system. In some non-limiting embodiments, power storage sub-system may include power source 14. In some non-limiting embodiments, power source 14 may include a direct current (DC) power source, such as one or more batteries. In some non-limiting embodiments, the electronics sub-system may control the actuation of a drug injection system (e.g., drug injection system 902) of drug delivery device 900. The electronics sub-system may include processing device 24, sensing electronics 26, pump and valve controller 28, sensing electronics 30, and deployments electronics 32. In some non-limiting embodiments, the fluidics sub-system may include reservoir 12, volume sensor 34 for reservoir 12, reservoir fill port 36, and metering sub-system 38, which includes pump and valve actuator 40 and pump and valve mechanism 42. The fluidic sub-system may further include occlusion sensor 44, deploy actuator 46, cannula 48 for insertion into the skin of a user, and fluid line 50 in fluid communication with reservoir 12 and cannula 48. In some non-limiting embodiments, drug injection system 902 may be configured to move cannula 48 from a retracted position positioned entirely within drug delivery device 900 to an extended position where cannula 48 extends outside of drug delivery device 900. In some non-limiting embodiments, cannula 48 is a catheter, which may be a soft, flexible catheter, a rigid catheter, or a combination thereof. In some non-limiting embodiments, drug delivery device 900 may operate in the same manner as discussed in U.S. Pat. No. 10,449,292 to Pizzochero et al., which is incorporated by reference herein in its entirety.

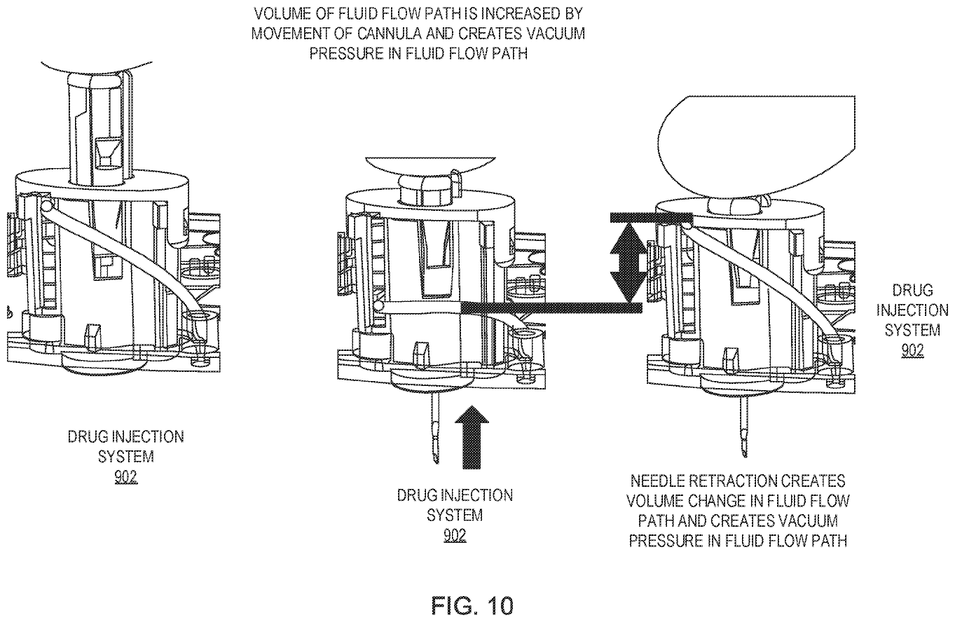

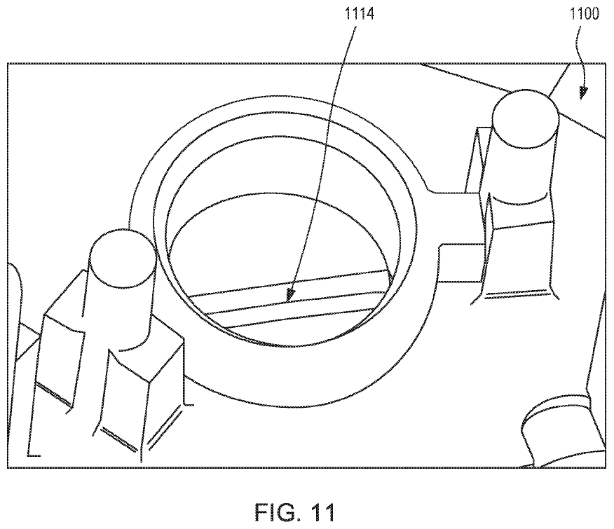

Referring now to FIG. 10, FIG. 10 is a diagram of implementation 1000 of drug injection system 902 of a drug delivery device during an injection procedure. In some non-limiting embodiments, implementation 1000 may include a user operating drug injection system 902, such that a drug may be delivered to the body of the user based on the user depressing a button (e.g., button 918) of drug injection system 902. As shown in FIG. 10, the cannula of drug injection system 902 may extend away from drug injection system 902 during an injection procedure based on the user depressing the button. As further shown in FIG. 10, a volume of a fluid flow path may increase and create vacuum pressure in the fluid flow path. The increase in volume of the fluid flow path may be based on movement of the cannula as well as movement of a needle of the cannula. As further shown in FIG. 10, the needle of the cannula may retract as part of the injection procedure. In some-non-limiting embodiments, a volume of the fluid flow path may change based on the needle retracting into the cannula, which may create vacuum pressure in the fluid flow path. The movement of the cannula and/or the needle may create a change in a characteristic associated with the fluid flow path that may be detected by a sensor device (e.g., sensor device 104).

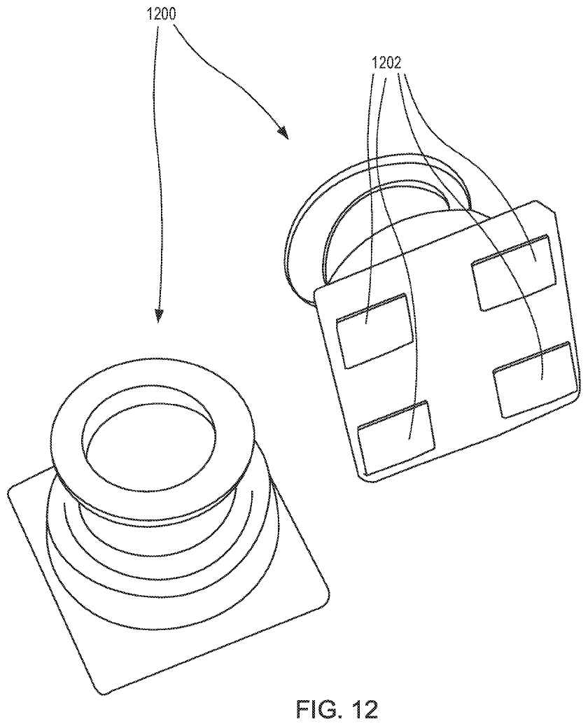

Referring now to FIG. 11, FIG. 11 is a diagram of base section 1100 of a drug delivery device (e.g., drug delivery device 900). As shown in FIG. 11, base section 1100 may include fluid flow path 1114. As further shown in FIG. 11, fluid flow path 1114 may be formed in base section 1100. In some non-limiting embodiments, base section 1100 may include a structure (e.g., a detent, a cavity, etc.) sized and configured to allow a sensor device, such as a pressure sensor, to be positioned within the structure and within fluid flow path 1114.

Referring now to FIG. 12, FIG. 12 is an illustration of sensor device 1200 of a drug delivery device (e.g., drug delivery device 900). In some non-limiting embodiments, sensor device 1200 may be the same as or similar to sensor device 104, flow rate sensing device 404, and/or pressure sensing device 504. As shown in FIG. 12, sensor device 1200 may include contact pins 1202. In some non-limiting embodiments, sensor device 1200 may include contacts pins 1202 to connect (e.g., electrically connect) sensor device 1200 to a printed circuit board (PCB). In some non-limiting embodiments, sensor device 1200 may include a pressure sensor and/or a temperature sensor. In some non-limiting embodiments, sensor device 1200 may include a pressure sensor that calculates pressure based on force applied to a contact surface of sensor device 1200. In some non-limiting embodiments, sensor device 1200 may have an operating pressure range of 4.3-17.4 pounds per square inch (psi) and/or a maximum pressure up to 65 psi. In some non-limiting embodiments, the pressure sensor may be an absolute pressure sensor with reading range of 0-30 bar absolute. In some non-limiting embodiments, the pressure sensor may be a gauge pressure sensor. In some non-limiting embodiments, sensor device 1200 may include a temperature range of −20 to 85 degrees Celsius. In some non-limiting embodiments, sensor device 1200 may communicate over a serial communication interface, such as an I²C interface paired with a multipoint control unit. In some non-limiting embodiments, sensor device 1200 may sense (e.g., detect, measure, record, etc.) pressure and/or temperature values in real-time.

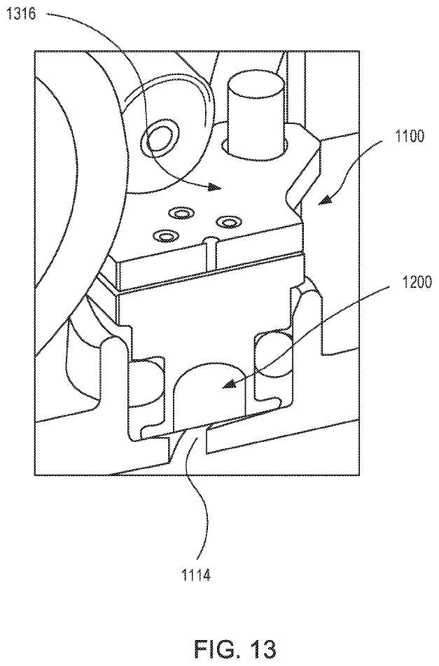

Referring now to FIG. 13, FIG. 13 is a diagram of sensor device 1200 positioned in base section 1100. As shown in FIG. 13, sensor device 1200 may be positioned in a structure of base section 1100, such that sensor device 1200 is positioned within fluid flow path 1114. In some non-limiting embodiments, sensor device 1200 may be connected (e.g., electrically) to PCB 1316 via contact pins 1202.

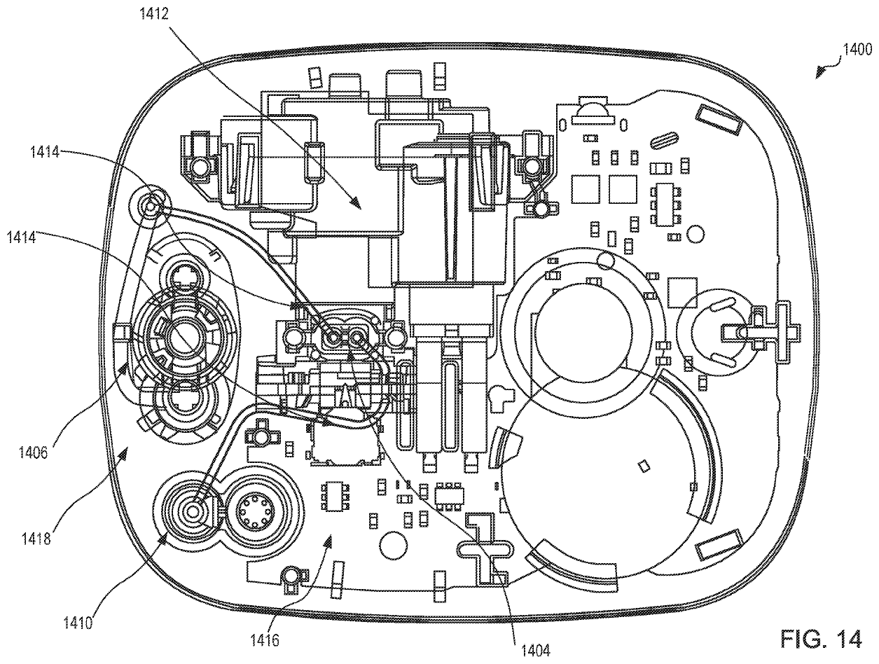

Referring now to FIG. 14, FIG. 14 is a plan view of drug delivery device 1400. In some non-limiting embodiments, drug delivery device 1400 may be the same as or similar to drug delivery device 100, drug delivery device 400, drug delivery device 500, and/or drug delivery device 900. As shown in FIG. 14, drug delivery device 1400 may include drug injection system 1402, sensor device 1404, reservoir 1410, pump device 1412, fluid flow path 1414, and PCB 1416. In some non-limiting embodiments, sensor device 1404 may be the same as or similar to sensor device 104, flow rate sensing device 404, pressure sensing device 504, and/or sensor device 1200. In some non-limiting embodiments, drug injection system 1406 may be the same as or similar to drug injection system 106, drug injection system 406, drug injection system 506, and/or drug injection system 902. In some non-limiting embodiments, reservoir 1410 may be the same as or similar to reservoir 110. In some non-limiting embodiments, pump device 1412 may be the same as or similar to pump device 112. As further shown in FIG. 14, sensor device 1404 may be positioned within fluid flow path 1414. In some non-limiting embodiments, one or more of sensor device 1404, drug injection system 1406, reservoir 1410, and/or pump device 1412 may be positioned on PCB 1416 or base section 1418.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A drug delivery device, for delivery of a drug, comprising:
at least one sensor; and
at least one processor in communication with the sensor, wherein the at least one processor is programmed or configured to:
receive data associated with a fluid flow path from the sensor during a time corresponding to a working mode of the drug delivery device;
determine a characteristic associated with the fluid flow path based on the data received from the sensor;
determine whether the characteristic associated with the fluid flow path satisfies at least one of a threshold and a characteristic time constant derived from a pressure decay; and
perform an action based on determining that the data associated with the fluid flow path satisfies the threshold at a time prior to, or upon completion of, delivery of a drug from the drug delivery device.

2. The drug delivery device of claim 1, wherein the sensor is a flow sensor.

3. The drug delivery device of claim 1, wherein the sensor is a pressure sensor.

4. The drug delivery device of claim 3, wherein the pressure sensor is positioned within the fluid flow path.

5. The drug delivery device of claim 1, wherein the at least one sensor is positioned in direct contact with the fluid path.

6. The drug delivery device of claim 1, further comprising:
a housing; and
a drug injection system;
wherein the at least one processor and the drug injection system are positioned within the housing.

7. The drug delivery device of claim 6, wherein the drug injection system comprises a cannula configured to be inserted into subcutaneous tissue or muscle tissue of a user.

8. The drug delivery device of claim 1, wherein the threshold comprises a threshold value based on a working mode of the drug delivery device.

9. The drug delivery device of claim 1, wherein the specific working mode of the drug delivery device is at least one of deployment of a component, injection of a drug, and dwell time.

10. A drug delivery device comprising:
a housing;
a drug injection system;
a sensor; and
at least one processor in communication with the sensor;
wherein the at least one processor, the sensor, and the drug injection system are positioned within the housing; and
wherein the at least one processor is programmed or configured to:
receive data associated with a fluid flow path from the sensor;
determine a characteristic associated with the fluid flow path based on the data received from the sensor;
determine whether the characteristic associated with the fluid flow path satisfies a threshold; and
perform an action based on determining that the data associated with the fluid flow path satisfies the threshold at a time prior to, or upon completion of, delivery of a drug from the drug delivery device.

11. The drug delivery device of claim 10, wherein the sensor is a flow sensor.

12. The drug delivery device of claim 10, wherein the sensor is a pressure sensor.

13. The drug delivery device of claim 12, wherein the pressure sensor is positioned within the fluid flow path.

14. The drug delivery device of claim 10, wherein the at least one sensor is positioned within the fluid path.

15. The drug delivery device of claim 10, further comprising:
a housing; and
a drug injection system;
wherein the at least one processor and the drug injection system are positioned within the housing.

16. The drug delivery device of claim 15, wherein the drug injection system comprises a cannula configured to be inserted into subcutaneous tissue or muscle tissue of a user.

17. The drug delivery device of claim 10, wherein the threshold comprises a threshold value based on a working mode of the drug delivery device.

18. A drug delivery device comprising:
at least one processor programmed or configured to:
receive data associated with a fluid flow path from a sensor;
determine a characteristic associated with the fluid flow path based on the data received from the sensor;
determine whether the characteristic associated with the fluid flow path satisfies a threshold; and
perform an action based on determining that the data associated with the fluid flow path satisfies the threshold;
wherein, when performing the action, the at least one processor is programmed or configured to:
provide an alarm;
provide a notification;
store data associated with an event; or
any combination thereof.

19. The drug delivery device of claim 18, wherein the sensor is positioned within the fluid flow path.

20. The drug delivery device of claim 18, further comprising:
a housing;
a reservoir;
a pump device;
a drug injection system; and
an adhesive layer;
wherein the at least one processor, the reservoir, the pump device, and the drug injection system are positioned within the housing; and
wherein the adhesive layer is configured to be positioned on a body of a user.

21. The drug delivery device of claim 20, wherein the drug injection system comprises a cannula configured to be inserted into subcutaneous tissue or muscle tissue of a user.

22. The drug delivery device of claim 18, wherein the action is performed based on determining that the data associated with the fluid flow path satisfies the threshold at a time prior to, or upon completion of, delivery of a drug from the drug delivery device.
